(19) United States
(12) Patent Application Publication
(10) Pub. No.: US 2003/0032913 A1
(43) Pub. Date: Feb. 13, 2003

(54) Intragraft dialysis catheter apparatus

(76) Inventor: Stanley Batiste, Las Vegas, NV (US)

Correspondence Address: HENDERSON & STURM LLP, 1213 MIDLAND BUILDING, 206 SIXTH AVENUE, DES MOINES, IA, 50309-4076, US

(21) Appl. No.: 09/925,274

(22) Filed: Aug. 9, 2001

(51) Int. Cl. A61M 37/00

(52) U.S. Cl. 604/5.01; 604/6.16

(57) ABSTRACT

An intragraft dialysis catheter apparatus (10) for installation in a pre-existing A-V dialysis shunt for prolonging the useful life of the A-V dialysis shunt for providing blood flow to the inlet and outlet ports of a conventional dialysis machine to cleanse a patient's blood supply in a well recognized manner. The apparatus includes a pair of generally thin walled reservoir members (30), (30′) each having a generally stiff backing plate member (40), (40′) covering at least the bottom portion of the respective reservoir members (30), (30′) and wherein each reservoir member (30), (30′) is in open fluid communication with one of a pair of elongated hollow catheter members (20), (20′).

### BACKGROUND OF THE INVENTION

1. Field of the Invention

The present invention relates to the field of surgical procedures and apparatus in general, and in particular to a dialysis catheter apparatus that are disposed in an intragraft disposition within a patient's arterial-venous system.

2. Description of Related Art

As can be seen by reference to the following U.S. Pat. Nos. 5,688,237; 6,056,717; 6,074,377; 6,095,997; and 6,099,513, the prior art is replete with myriad and diverse catheter associated accessories.

While all of the aforementioned prior art constructions are more than adequate for the basic purpose and function for which they have been specifically designed, they are uniformly deficient with respect to their failure to provide a simple, efficient, and practical approach to solving the problem addressed by the intragraft dialysis catheter apparatus of the present invention.

Patients with end stage renal disease (chronic renal failure) on hemodialysis pose very difficult vascular management problems. Hemodialysis involves the filtering of unwanted products out of the blood which are normally removed by the kidneys. Several methods for dialysis are currently available, these include peritoneal dialysis (intraabdominal), external vascular access catheters, most commonly A-V fistulas/grafts and reservoir type catheters. Maintaining access into the vascular system is necessary for the long term survival of these patients.

Generally, the course of management for dialysis patients involves the placement of an arterial-venous shunt (A-V shunt). These are surgically placed initially within the forearm, when these eventually fail grafts are then placed within the upper arm followed by the groin and in some cases, across the upper chest. Either a synthetic graft is used or a direct fistula is created in which the artery is connected directly onto the native vein or graft. These A-V shunts then allow large volumes of blood delivered via the artery to be “shunted” across the graft or fistula into the central venous system. As the shunts mature and enlarge, they can then be easily accessed by the dialysis staff. Access will be made into the proximal end for the aspiration of blood and the venous end for blood return.

The problem with this method is that the shunts usually do not stay open for more than 2.5 years and many last only several months due to scarring and clotting. The patient will then require another shuts to be placed. The old occluded shunt will then remain non functional where it was initially placed. Each shunts therefore uses up patient's valuable access until shunt access is exhausted. At this point current management is to place external catheters which are prone to infection and are considered cosmetically very unappealing as they are placed within neck veins.

This proposed design will allow continued access through a previously occluded graft that up until now has been rendered useless by scarring and clotting. Simply the design involves two separate reservoir chambers, each attached to individual catheters (see FIGS. 5 and 6). The proximal reservoir will allow blood to be withdrawn and the distal reservoir will allow blood return. These reservoirs will be easily placed within the old non functioning dialysis shunt. The individually attached catheters will be positioned with the tips within the central venous system. This system can be easily placed under local anesthesia and will have the following significant benefits: the entire reservoir/catheter system will be beneath the skin and will therefore be at much less risk for infection and damage than external catheters; the access provided by dialysis centers will not differ from the usual A-V shunt access procedure, thus limiting further training and potentially reducing complications; cosmetically, this eliminates the need for the unappealing appearance of external catheters; most significantly, this design will greatly increase the duration of patient's vascular access and will therefore likely significantly increase patient long term survival.

As a consequence of the foregoing situation, there has existed a longstanding need for a new and improved intragraft dialysis catheter apparatus, and the provision of such a construction is a stated objective of the present invention.

### BRIEF SUMMARY OF THE INVENTION

Briefly stated, the intragraft dialysis catheter apparatus that forms the basis of the present invention comprises in general a pair of catheter units each provided with a reservoir unit and a barrier plate unit, wherein the catheter units are operatively associated with one another.

As will be explained in greater detail further on in the specification, each of the reservoir units comprises a generally think walled reservoir member adapted to be accessed by either the inlet or outlet port of a dialysis machine and the barrier plate units each comprises a generally stiff barrier plate member that covers at least the bottom half of one of the reservoir members to prevent the dialysis access couplings from passing through the respective reservoir members.

Furthermore, the intragraft dialysis catheter apparatus comes in a combined straight line version for use in a straight line A-V dialysis shunt and a spaced piggyback version for use in a loop typ A-V dialysis shunt. Both versions are structurally related and designed to remedy the problem with the shunts failing from scarring and accumulated blood clots as described earlier on.

### BRIEF DESCRIPTION OF THE SEVERAL VIEWS OF THE DRAWINGS

These and other attributes of the invention will become more clear upon a thorough study of the following description of the best mode for carrying out the invention, particularly when reviewed in conjunction with the drawings, wherein:

FIG. 1 depicts a straight type A-V dialysis shunt installed in a patient's arm;

FIG. 2 is a cross sectional view of a patient's arm showing a superficially placed A-V graft;

FIG. 3 depicts a loop type A-V dialysis shunt installed in a patient's arm;

FIG. 4 depicts an occluded shunt in a patient's arm with scarring and blood clots;

FIG. 5 is a cross-sectional view of the co-joined version of the intragraft dialysis catheter apparatus wherein FIGS. 5A through 5C show representative spaced cross sectional views along the length of this version;

FIG. 6 is a cross sectional view of the spaced piggy pack version of the apparatus wherein FIGS. 6A through 6D show representative spaced cross sectional view along the length of this version;

FIG. 7 depicts the co-joined version of the apparatus installed in a straight A-V dialysis shunt; and

FIG. 8 depicts the spaced piggy back version of the apparatus installed in a loop type A-V dialysis shunt.

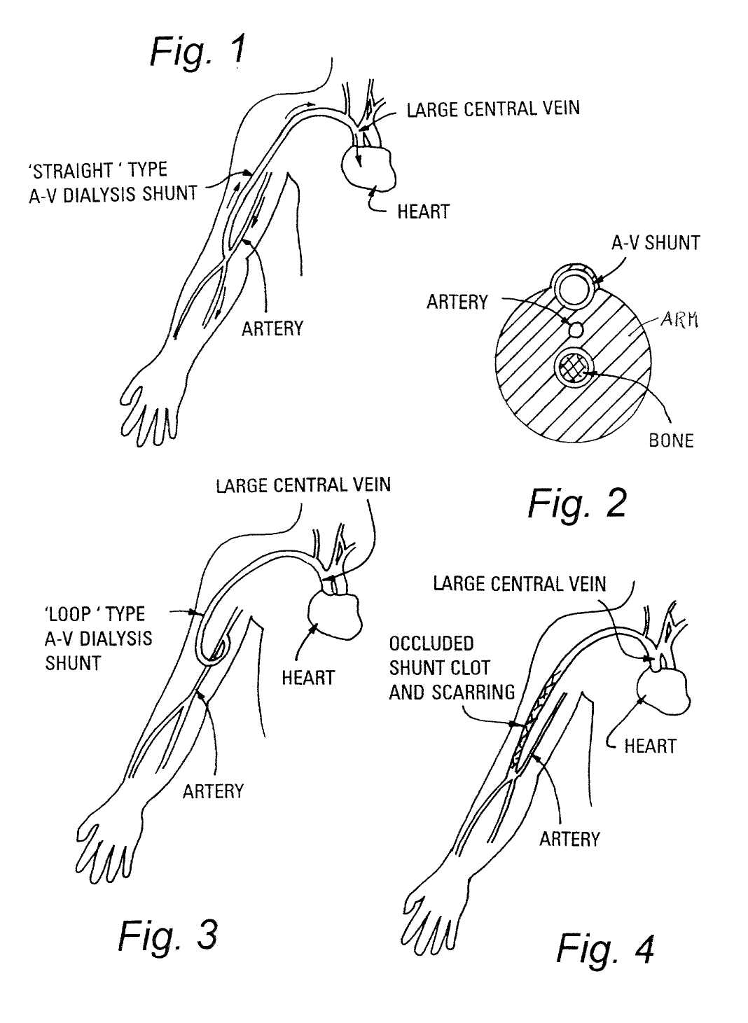

### DETAILED DESCRIPTION OF THE INVENTION

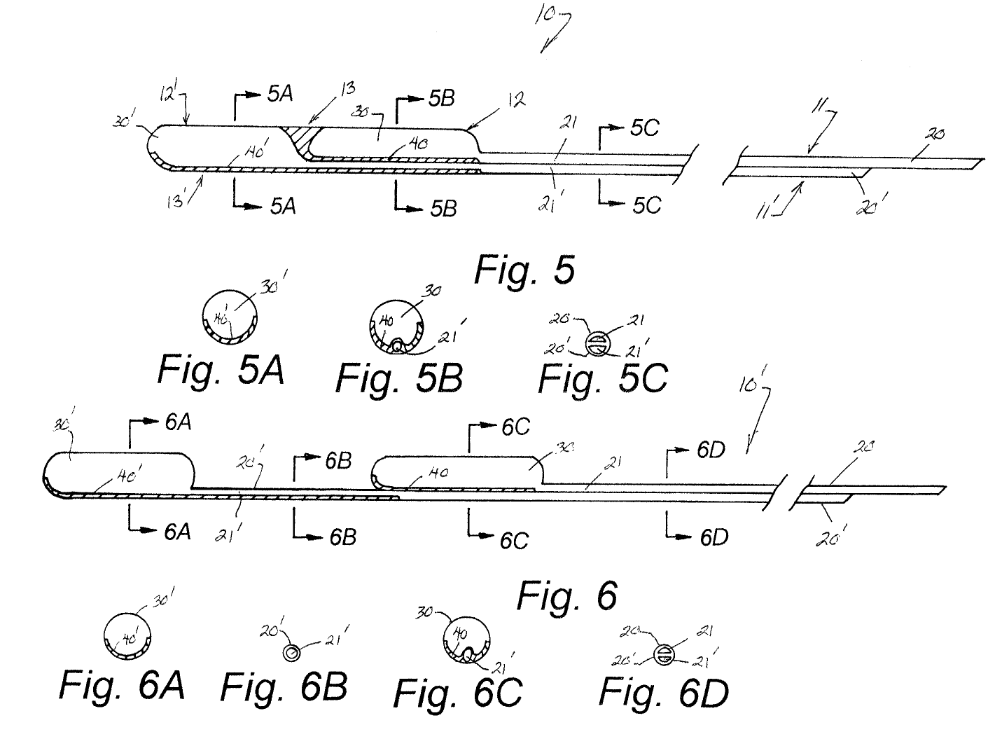

As can be seen by reference to the drawings, and in particular to FIGS. 5 and 6, the intragraft dialysis catheter apparatus that forms the basis of the present invention is designated generally by the reference number 10. The apparatus 10 comprises in general a pair of catheter units 11, 11′ which are each provided with a reservoir unit 12 and 12′ and a barrier plate unit 13 and 13′. These units will now be described in seriatim fashion.

As shown in FIGS. 5 and 6, as well as their respective sub-figures, each of the catheter units 11 and 11′ comprise an elongated hollow tubular catheter member 20, 20′ which are joined together over a substantial portion of their length as depicted in FIGS. 5, 5C, 6, and 6D. Each catheter member 20, 20′ defines a relatively narrow hollow passageway 21, 21′ which is in open communication with the interior of one of the reservoir units 12, 12′.

In the co-joined version of the apparatus 10 shown in FIG. 5, and FIGS. 5A through 5C, the reservoir units 12, 12′ comprise respective distal 30 and proximal 30′ enlarged generally thin walled reservoir members which are disposed adjacent to one another. Whereas, in the piggy back version of the invention depicted in FIG. 6 and FIGS. 6A through 6D, the distal 30 and proximal 30′ reservoir members are relatively widely spaced with respect to one another for reasons that will become apparent further on in the specification.

Returning once more to FIG. 5, and FIGS. 5A and 5B, it can be seen that in the co-joined version of the apparatus each of the reservoir members 30, 30′ are further provided with a barrier plate unit 13, 13′. Each barrier plate unit 13, 13′ comprise a reinforced barrier plate member 40, 40′ which covers the lower half of each reservoir member 30, 30′.

Furthermore, in the co-joined version of the apparatus 10, the distal barrier plate member 40 forms a common wall for the distal end of the distal reservoir 30 and the proximal end of the proximal reservoir 30′, as well as a portion of the catheter member 20′.

In addition, barrier plate member 40′ extends along a portion of catheter member 20′ beneath the distal reservoir 30.

However, as depicted in FIGS. 6 and 6A through 6C, the barrier plate members 40 and 40′ are independent structural components within their respective reservoir members 30, 30′, but barrier plate member 40′ does extend a distance along the catheter member 20′ to a point beneath the leading edge of the distal reservoir 30.

At this juncture, it should be noted that the barrier plate members 40, 40′ are provided primarily to protect the patient's arm and to prevent the puncturing of the bottom portions of the proximal 30′ and distal 30 reservoirs.

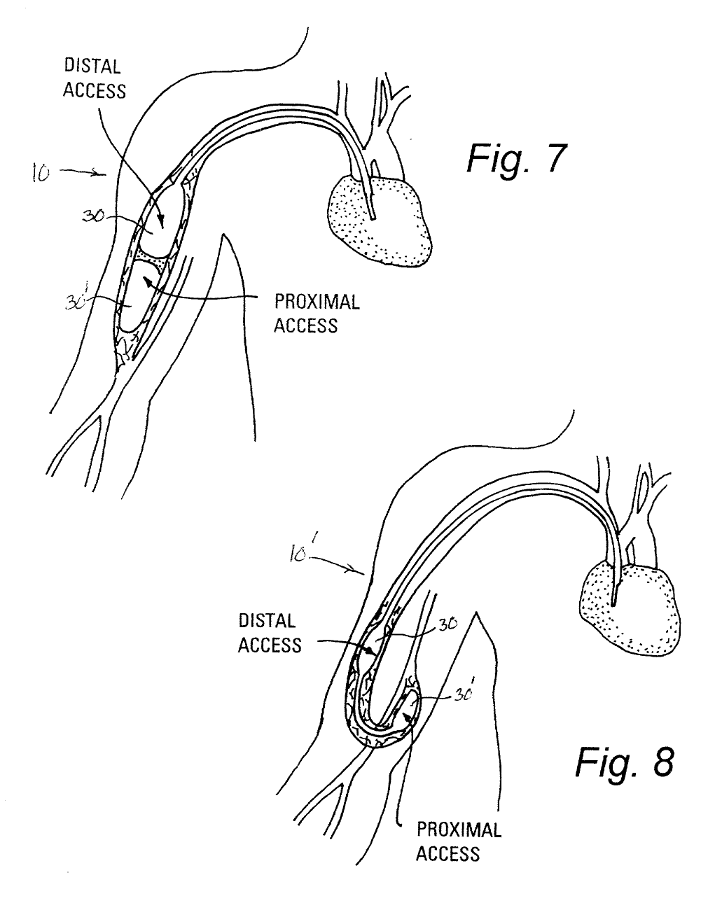

Turning now to FIG. 7, it can be seen that the co-joined version of the apparatus 10 is specifically designed to be installed in a straight A-V dialysis shunt 100. Whereas, as shown in FIG. 8, the spaced piggy back version of the apparatus 10′ is specifically designed to be installed in a loop type A-V dialysis shunt.

Although only an exemplary embodiment of the invention has been described in detail above, those skilled in the art will readily appreciate that many modifications are possible without materially departing from the novel teachings and advantages of this invention. Accordingly, all such modifications are intended to be included within the scope of this invention as defined in the following claims.

Having thereby described the subject matter of the present invention, it should be apparent that many substitutions, modifications, and variations of the invention are possible in light of the above teachings. It is therefore to be understood that the invention as taught and described herein is only to be limited to the extent of the breadth and scope of the appended claims.

I claim:

1. An intragraft dialysis catheter apparatus for installation in an existing A-V dialysis shunt wherein the apparatus comprises:
a catheter unit including a catheter member defining an elongated hollow passageway;
a reservoir unit including an enlarged relatively thin walled reservoir member in open fluid communication with said catheter member; and
a barrier plate unit operatively associated with said reservoir unit and including a barrier plate member which covers the lower half of the reservoir member.

2. An intragraft dialysis catheter apparatus for installation in an existing A-V dialysis shunt wherein the apparatus comprises:
a pair of catheter units wherein each of the catheter units includes a catheter member defining an elongated hollow passageway wherein the catheter members are connected to one another along at least a substantial portion of their respective lengths;
a pair of reservoir units wherein each reservoir unit includes an enlarged generally thin walled reservoir member disposed in open fluid communication with one of said catheter members; wherein each reservoir member is adapted to be operatively connected to a selective one of a conventional dialysis machines inlet and outlet access ports.

3. The apparatus as in claim 2 further comprising a pair of barrier plate units wherein each barrier plate unit includes a generally stiff barrier plate member that covers at least the bottom portion of one of the reservoir units.

4. The apparatus as in claim 3 wherein the reservoir members are co-joined together and one of the barrier plate members forms a common wall between the reservoir members.

5. The apparatus as in claim 3 wherein the reservoir members are physically spaced from one another in a piggy pack fashion.

6. The apparatus as in claim 4 wherein the apparatus is adapted to be installed in a straight type A-V dialysis shunt.

7. The apparatus as in claim 5 wherein the apparatus is adapted to be installed in a loop type A-V dialysis shunt.

8. A method of prolonging the useful life of an A-V dialysis shunt extending from a patient's large central vein to a selected artery wherein the dialysis shunt is intended to circulate blood to and from the inlet and outlet access ports of a conventional dialysis machine comprising the steps of:
(a) inserting an intragraft dialysis catheter apparatus into the A-V dialysis shunt wherein the apparatus comprises a pair of generally thin walled enlarged reservoir members threadedly disposed within the A-V dialysis shunt via individual elongated hollow catheter members wherein each reservoir member is provided with a generally stiff backing plate member that covers at least the bottom portion of the respective reservoir member;
(b) establishing open fluid communication through the walls of A-V shunt and into the interior of one of the reservoir members with the inlet of the dialysis machine; and
(c) establishing open fluid communication through the wall of the A-V dialysis shunt and into the interior of the other of the reservoir members with the outlet of the dialysis machine.
